(19) United States
(12) Patent Application Publication
(10) Pub. No.: US 2018/0155290 A1
(43) Pub. Date: Jun. 7, 2018

(54) Improved Process for the Preparation of Aripiprazole with Reduced Particle Size

(71) Applicant: Davuluri Ramamohan RAO, Hyderabad (IN)

(72) Inventors: Ravi PONNAIAH, Madurai (IN); Kumar NEEL PRAVEEN, Hyderabad (IN); Guruswamy BATTHINI, Hyderabad (IN); Venkata Narayana GURRAM, Hyderabad (IN); Naresh DONGARI, Nalgonda (IN)

(21) Appl. No.: 15/572,492

(22) PCT Filed: Dec. 14, 2015
(86) PCT No.: PCT/IN2015/000460

§ 371 Date: Nov. 7, 2017

(30) Foreign Application Priority Data
May 8, 2015 (IN) 2339/CHE/2015

(51) Int. Cl. C07D 215/227

(52) U.S. Cl. CPC C07D 215/227

(57) ABSTRACT

The present invention relates to process for preparation of Aripiprazole with reduced particle size having dehydro impurity less than 0.1%.

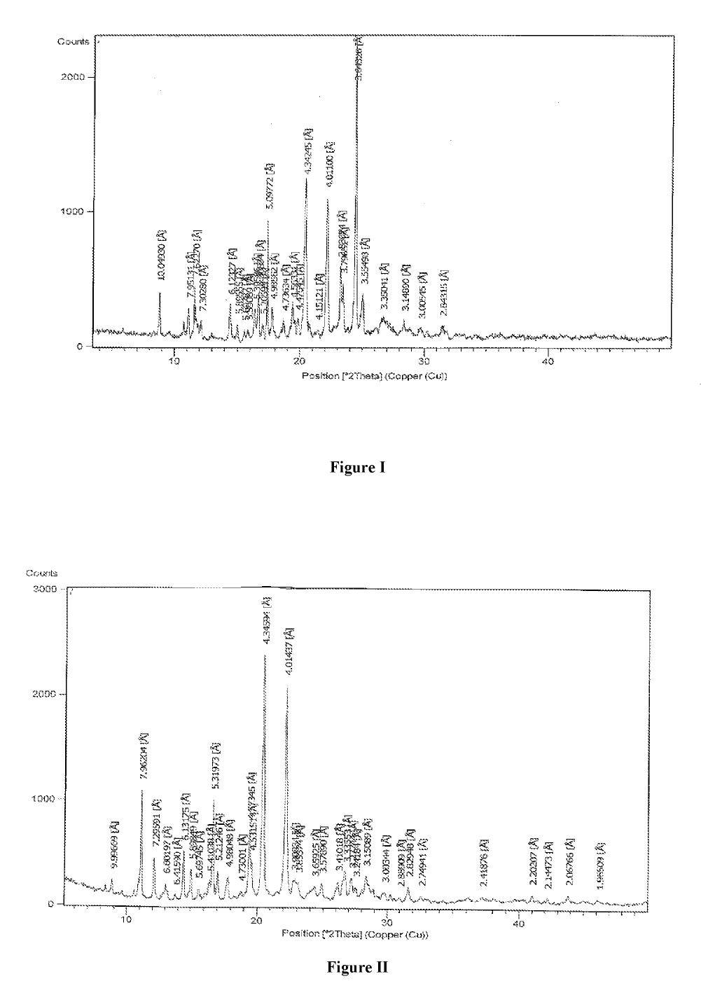

### FIELD OF THE INVENTION

The invention relates to an improved process for the preparation of Aripiprazole having formula (I)

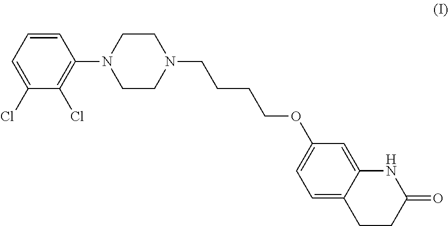 [SMILES: O=C1CCc2ccc(OCCCCN3CCN(c4cccc(Cl)c4Cl)CC3)cc2N1]

The invention also relates to processes for the preparation of Aripiprazole with reduced particle size.

### BACKGROUND OF THE INVENTION

Aripiprazole is chemically known as 7-[4-[4-(2,3-dichlorophenyl)-1-piperazinyl]butoxy]-3,4-dihydrocarbostyril having the formula (I)

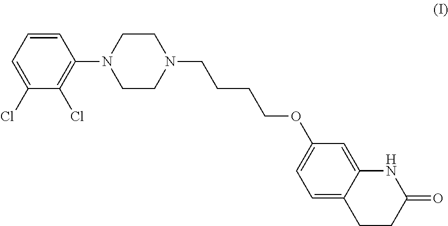 [SMILES: O=C1CCc2ccc(OCCCCN3CCN(c4cccc(Cl)c4Cl)CC3)cc2N1]

Aripiprazole is atypical antipsychotic agent useful for the treatment of schizophrenia. Aripiprazole is marketed as oral tablets under the trade name of Abilify®.

U.S. Pat. No. 5,006,528, US20070032651, WO2003026659, WO20040663162 and WO2006079549 describe process for the preparation of Aripiprazole by using 7-hydroxy-3,4-dihydrocarbostyril as intermediate. The Aripiprazole obtained from these prior art reference having unwanted dehydro impurity of formula (II)

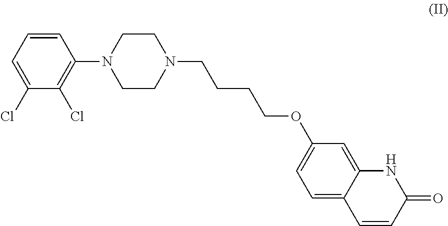 [SMILES: O=c1ccc2ccc(OCCCCN3CCN(c4cccc(Cl)c4Cl)CC3)cc2[nH]1]

WO 2006038220A1 discloses process for the preparation of Aripiprazole from novel intermediate of 6-Hydroxy 1-indanone. This process requires separate preparation of novel intermediate, which make this process more tedious.

In summary, process disclosed in prior art references for the preparation of Aripiprazole are tedious and requires laborious column chromatography for removal of unwanted impurities which causes low yield and purity.

Moreover, the prior art process yield Aripripazole with larger particle size. For controlled release sterile Aripiprazole formulation, there is need of 95% particles with particle size≤100 microns. The required particle size obtained from micronization techniques causes change in polymorphic form.

Therefore, there still exists need for methods for reducing average particle size with cost effective and safer process for large scale production of Aripripazole.

### OBJECTS OF THE INVENTION

Primary object of the invention is to provide an improved process for the preparation of Aripripazole.

Another object of the invention is to provide a simple and cost effective process for the preparation of Aripripazole having dehydro impurity less than 0.1%.

Another object of the invention is to provide the process for preparation of Aripiprazole with average particle size less than 35 μm.

### SUMMARY OF THE INVENTION

In one aspect, the present invention provided process for preparation of Aripiprazole having dehydro impurity less than 0.1%.

In another aspect, the present invention provides improved process for the preparation of Aripiprazole of formula (I)

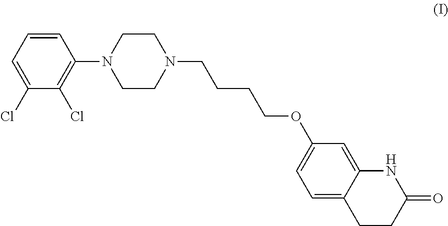 [SMILES: O=C1CCc2ccc(OCCCCN3CCN(c4cccc(Cl)c4Cl)CC3)cc2N1]

which comprises:

i) reacting 1-(2,3-dichlorphenyl)piperazine or its salt of formula (III)

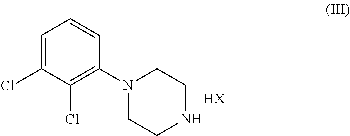

with 1,4-dibromobutane to obtain spiro compound or its salt of formula (IV)

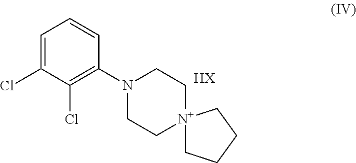

ii) the spiro compound or its salt of formula (IV) treated with the 7-hydroxy-3,4-dihydroquinoline-2(1H)-one or its salt of formula (V)

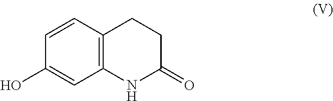 [SMILES: O=C1CCc2ccc(O)cc2N1]

in polar aprotic solvent and non-polar solvent mixture to obtain the compound of formula (I) of Aripripazole.

In another aspect, the present invention provides process for purification of Aripiprazole

which comprises:

i) dissolve Aripiprazole in aprotic solvent,

ii) heat the step i) reaction mixture,

iii) cool the reaction mixture,

iv) add polar solvent to precipitate,

v) heat the obtained step iv) precipitate,

vi) filter the compound in hot condition.

In another aspect, the present invention provides process for preparation of Aripiprazole with average particle size less than 35 μm.

which comprises:

i) dissolve Aripiprazole in polar solvent,

ii) reflux step i) reaction mixture,

iii) add step ii) reaction mixture to pre-cooled ethanol,

iv) cool reaction mixture,

v) filter the obtained product.

### DRAWINGS

FIG. I: XPRD of the Aripiprazole.

FIG. II: XPRD of the Aripiprazole after reducing the particle size.

### DETAILED DESCRIPTION OF THE INVENTION

Accordingly, the present invention provides a process for the preparation of Aripiprazole of formula (I), Scheme I illustrates the process for the preparation of formula (I).

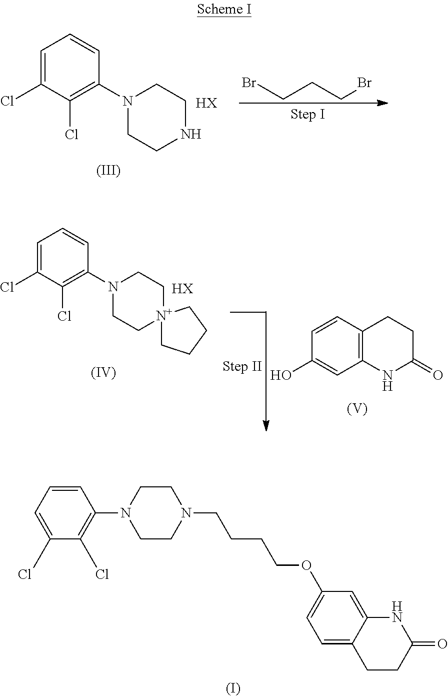

which comprises:

Step I) reacting 1-(2,3-dichlorphenyl)piperazine or its salt of formula (III) with 1,4-dibromobutane to obtain spiro compound or its salt of formula (IV),

Step II) the spiro compound or its salt of formula (IV) treated with the 7-hydroxy-3,4-dihydroquinoline-2(1H)-one or its salt of formula (V) in polar aprotic solvent and non polar solvent mixture to obtain the compound of formula (I) of Aripripazole, optionally further crystallized in polar solvent.

The polar aprotic solvent is selected from dimethyl sulfoxide, dimethyl formamide, 1-methyl-2-pyrrolidinone, hexamethylphosphoramide and hexamethyl phosphorus triamide, and preferably dimethyl sulfoxide. The non-polar solvent is selected from cyclic hydrocarbons like toluene, and preferably toluene.

According to another aspect of the invention, the process for purification of Aripiprazole

which comprises:

i) dissolve Aripiprazole in aprotic solvent,

ii) heat the step i) reaction mixture,

iii) cool the reaction mixture,

iv) add polar solvent to precipitate,

v) heat the obtained step iv) precipitate up to dissolve,

vi) filter the compound in hot condition.

The polar aprotic solvent of step i) is selected from dimethyl formamide, 1-methyl-2-pyrrolidinone, hexamethylphosphoramide and hexamethyl phosphorus triamide, and preferably dimethyl sulfoxide.

The polar protic solvent of step iv) is selected from alcohols such as ethanol, isopropyl alcohol, isobutyl alcohol and tertiary-butyl alcohol, Preferably methanol.

The reaction temperature of step ii) may range from 50° C. to 80° C. and preferably at a temperature in the range of 60° C. to 70° C. The duration of the reaction may range from 1 hour to 2 hours, preferably for a period of 1 hour.

According to another aspect of the invention, the process for preparation of Aripiprazole with average particle size less than 35 μm.

which comprises:

i) dissolve Aripiprazole in polar solvent,

ii) reflux step i) reaction mixture,

iii) add step ii) reaction mixture to pre-cooled ethanol,

iv) cool reaction mixture,

v) filter the obtained product.

The duration of reaction reflux of step ii) may range from 30 minutes to one hour, preferably for a period of 30 minutes.

Some aspects of this disclosure are described in the examples below, which are provided only for the purpose of illustration and are not intended to limit the scope of the disclosure in any manner.

Experimental Procedures

### EXAMPLE-1

### Process for the Preparation of Compound of Formula IV (Quaternary Spiroammonium Salt)

The 1-(2,3-dichlorphenyl)piperizine hydrochloride (200 grams) was dissolved in acetone (1000 mL) and added potassium carbonate (206 grams). The reaction mixture was stirred for 15 minutes at room temperature and add 1,4-dibromobutane (111 grams), slowly rise the temperature up to 50±5° C. and stir for one hour at same temperature. The reaction mixture was cooled to room temperature, filtered and washed with acetone. The solid, thus obtained was dried (530 grams).

¹H NMR 300 MHz (D2O, δ ppm): 6.95 (m, 3H), 3.42 (s, 4H), 3.39 (s, 4H), 3.02 (s, 4H), 2.02 (s, 4H), 13C NMR: 148.76, 133.44, 128.61, 126.89, 126.04, 119.96, 63.33, 60.00, 46.67, 21.54.

### EXAMPLE-2

### Process for the Preparation of Aripiprazole

The compound of Example-1 (150 grams) is dissolved in toluene (600 mL) and dimethyl sulfoxide (50 mL) and add 7-hydroxy-3,4-dihydro-quinolinone (29 grams). The reaction mixtures were stirred for 30 minutes at room temperature and add potassium carbonate. Slowly raise the temperature of the reaction mixture and reflux at 100-115° C. for 7 hours. After the completion of reaction, cool the reaction up to 10-15° C. and add water (300 mL). The reaction mixture was stirred for two hour at 0-10° C. The precipitated compound was washed with toluene to obtain the compound (58 grams). Purity (HPLC): 98.44%. Dehydro impurity (HPLC)˜0.15%.

### EXAMPLE-3

### Purification of Aripiprazole in Dimethylsulfoxide and Methanol

The compound of Example-2 (50 grams) was dissolved in dimethylsulfoxide (100 mL) at room temperature. Slowly rise the reaction temperature up to 65-70° C. and stir for 45 minutes at same temperature. The reaction mixture was cooled to 50-55° C. and add methanol (400 mL) at same temperature. The reaction mixture is heated up to 65-70° C. and stirred for 45 minutes at same temperature. The reaction mixture was cooled to 40-42° C. and stir for one hour at same temperature. The precipitated compound was filtered and washed with methanol (200 mL) to obtain the compound (45 grams). Purity (HPLC): 99.75%. Dehydro impurity (HPLC)˜0.04%.

### EXAMPLE-4

### Purification of Aripiprazole

The Aripripazole (58 grams) from Example-2 was dissolved in ethanol (580 mL) at room temperature. Slowly raised the reaction mixture up to refluxed temperature and stirred for 15 minutes. The reaction mixture cooled, filtered the precipitated compound and wash with hot ethanol (52 grams) to obtain the compound (54 grams). Purity (HPLC): 99.99%.

### EXAMPLE-5

### Process for the Preparing Aripiprazole with Reduced Particle Size

The Aripiprazole (58 grams) from Example-2 was dissolved in ethanol (580 mL) at room temperature. Slowly raised the reaction mixture up to refluxed temperature and added activated carbon. The reaction mixture stirred for 15 minutes. The reaction mixture filtered in hot condition to obtain clear Aripiprazole solution. This clear solution added to pre-cooled (0-5° C.) ethanol (230 mL) solution. This reaction mixture is cooled to 0-10° C. for one hour. The precipitated compound filtered and washed with hot ethanol (52 grams) to obtain the compound (54 grams). Purity (HPLC): 99.99%.

Particle Size Distribution:

| Batch No | d(10) μm | d(50) μm | d(90) μm |
|---|---|---|---|
| 1. | 9.7 | 24.61 | 45.58 |
| 2. | 12.77 | 31.96 | 59.78 |
| 3. | 12.99 | 32.00 | 59.95 |

What is claimed is:

1. A process for the preparation of Aripiprazole of formula (I) 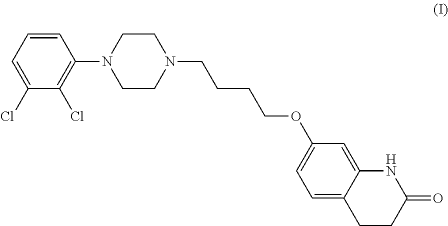 [SMILES: O=C1CCc2ccc(OCCCCN3CCN(c4cccc(Cl)c4Cl)CC3)cc2N1]
which comprises:
i) reacting 1-(2,3-dichlorphenyl) piperazine or its salt of formula (III) 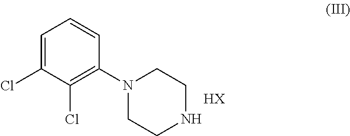
with 1,4-dibromobutane to obtain spiro compound or its salt of formula (IV) 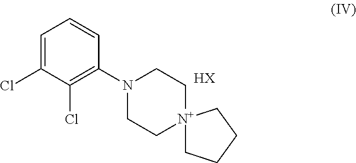
ii) the spiro compound orits salt of formula (IV) treated with the 7-hydroxy-3,4-dihydroquinoline-2(1H)-one or its salt of formula (V) 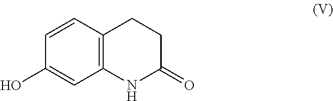 [SMILES: O=C1CCc2ccc(O)cc2N1]
in the polar aprotic solvent and non-polar solvent mixture to obtain Aripripazole of formula (I).

2. The process as claimed in claim 1, wherein polar aprotic solvent is dimethyl sulfoxide.

3. The process as claimed in claim 1, wherein non polar solvent is toluene.

4. A process for purification of Aripiprazole
which comprises:
i) dissolve Aripiprazole in aprotic solvent,
ii) heat the step i) reaction mixture,
iii) cool the reaction mixture,
iv) add polar solvent to precipitate,
v) heat the obtained step iv) precipitate up to dissolve,
vi) filter the compound in hot condition.

5. The process as claimed in claim 4, wherein polar aprotic solvent of step i) is selected from dimethyl formamide, 1-methyl-2-pyrrolidinone, hexamethylphosphoramide, hexamethyl phosphorus triamide or dimethyl sulfoxide.

6. The process as claimed in claim 5, wherein polar aprotic solvent of step i) is dimethyl sulfoxide.

7. The process as claimed in claim 4, wherein polar solvent of step iv) is selected from methanol, ethanol, isopropyl alcohol, isobutyl alcohol or tertiary-butyl alcohol.

8. The process as claimed in claim 7, wherein polar solvent of step iv) is selected from methanol.

9. A process for preparation of Aripiprazole having with average particle size less than 35 μm.
which comprises:
i) dissolve Aripiprazole in polar solvent,
ii) reflux step i) reaction mixture,
iii) add step ii) reaction mixture to pre-cooled ethanol,
iv) cool reaction mixture,
v) filter the obtained product.

10. The process for preparation of Aripiprazole as claimed in claim 1 having dehydro impurity less than 0.1%.

11. The process as claimed in claim 4, wherein polar aprotic solvent of step i) is dimethyl sulfoxide.

12. The process as claimed in claim 4, wherein polar solvent of step iv) is selected from methanol.
